(19) United States
(12) Patent Application Publication
(10) Pub. No.: US 2026/0272282 A1
(43) Pub. Date: Sep. 17, 2026

(54) APPARATUS HAVING A SCANNING DEVICE

(71) Applicant: Heidelberg Engineering GmbH, Heidelberg (DE)

(72) Inventor: Christoph BROSCHE, Eppelheim (DE)

(73) Assignee: Heidelberg Engineering GmbH, Heidelberg (DE)

(21) Appl. No.: 19/471,141

(22) PCT Filed: May 16, 2024
(86) PCT No.: PCT/EP2024/063502

§ 371 Date: Sep. 30, 2025

(30) Foreign Application Priority Data
Jul. 6, 2023 (DE) 10 2023 117 847.4

(51) Int. Cl. A61B 3/10

(52) U.S. Cl. CPC A61B 3/102

(57) ABSTRACT

The invention relates to an apparatus (10, 10′) for examining an object or for scanning an eye (1), comprising a scanning device (2, 2a) having a reflective surface (3) on which at least one light beam (4) emitted by a light source (8) can be deflected, it being possible for a light beam (5a) reflected by the reflective surface (3), and emanating towards the incident light beam (4), to be pivoted by the scanning device (2) over a first angular range (6a), characterised in that optical means are provided, by means of which the emanating reflected light beam (5a) can be pivoted by moving the means through a second angular range (6c) that is greater than the first angular range (6a).

The invention relates to an apparatus according to the preamble of claim 1.

An apparatus for scanning an eye usually comprises a scanning device with a scan area or reflection area, onto which an incident light beam can be steered, with a light beam reflected due to the incident light beam off the reflection area being pivotable over an angular range, the so-called scan angle, by means of the scanning device. This allows structures to be examined by scanning with light beams.

Against this background, confocal laser scanning systems with a beam and a point detector are already known. Moreover, laser scanning systems with line illumination and line detection are known.

Conventional confocal laser scanning systems are limited in terms of luminous intensity and speed. This is due to the fact that the properties of scan speed, scan angle and scan area depend on one another to a certain extent and cannot be increased to any desired extent.

Scanning devices with large scan areas and scan angles are often comparatively slow. Conversely, small scanning devices with small scan angles are often very fast.

The product of scan area or reflection area and scan angle is decisive for the luminous intensity and cannot be increased even by way of optical conversions. Especially for wide-angle systems, the luminous intensity or speed might be limited. Scanning devices with small scan angles use long focal lengths in order to create large intermediate images. This increases the space required for an optical system.

Laser scanning systems with line illumination and line detection are confocal only in one axis. This is accompanied by a lower image quality and relatively complicated optics, and very sensitive line-scan cameras are required. Additional optics are required as soon as multiple detectors and light sources are used or the beam expander route is followed, and this is associated with higher costs.

The problem addressed by the present invention is therefore that of providing an apparatus having a scanning device that allows a large overall scan angle to be realized as easily as possible, in particular without reducing the scan area or the speed.

The present invention solves the aforementioned problem by way of the features of claim 1.

First of all, it has been recognized-in inventive fashion-that there is a need for wide-angle scanning systems, in particular of +30° and more, in front of the eye, which systems are of high light-gathering power, are quick and have a very good image quality. It has also been recognized that these wide-angle scanning systems should be as compact as possible in order to be operational as intended. It has also been recognized that there is a need for fast scanning systems with smaller scan angles.

According to the invention, it has been recognized that an apparatus for examining an object or for scanning an eye must comprise at least one scanning device having a reflection area, onto which at least one light beam emitted by a light source can be steered, with a reflected light beam emanating from the reflection area due to the incident light beam being pivotable over a first angular range by means of the scanning device.

Optical means are provided in inventive fashion, by means of said optical means the outgoing reflected light beam is pivotable over a second angular range after passing through said means, wherein the second angular range is larger than the first angular range. What this realizes is that a light beam, in particular a laser beam, runs multiple times over the same scan area or reflection area of the scanning device, albeit from different angles, like in a loop, such that the overall scan angle is increased.

The light beam is deflected via beam-guiding components and then imaged on the same scan area or reflection area again, or the scanning device is optically imaged on itself, as it were. The scan angle is increased, preferably doubled, while the scan area remains the same. This multiplies the product from area by angle, whereby systems with higher luminous intensity or speed may be realized.

The apparatus described here enables a more flexible scanner selection. A larger scan angle allows for wide-angle systems to get closer to 1:1 imaging, which on account of optical symmetry is an advantage in the context of the formation of certain optical aberrations. A low optical scaling factor results in shorter focal lengths, which in turn has a positive effect on the spatial requirements.

The second angular range could be twice the size of the first angular range. This increases the overall scan angle particularly effectively and optimizes its magnification.

The reflected light beam emanating from the reflection area could be guided from the optical means back to the reflection area and could be pivoted from there over the second angular range as an actively returned light beam. The light beam, which is pivoted over the second angular range, is then a twice scanned light beam. The scanning device or its scan area is thus, as it were, deflected via optical components and imaged onto itself. The light which should be scanned or detected passes multiple times over the scanning device, preferably over the same place or the same surface, and is thus scanned multiple times. This increases the overall scan angle of the scanning system, wherein the beam diameter of the light beam does not change, thereby substantially maintaining its luminous intensity. Specifically, the beam diameter remains the same, and the luminous intensity is increased since the scan angle is increased. The product of the scan angle and scan area is thus increased by the use of optical deflectors.

The light beam incident from the light source, a standing light beam as it were, could be guided to the reflection area at a different angle than the actively returned light beam. The size of the overall scan angle can be set by the choice of the other angle.

After passing through the optical means and a further incidence on the reflection area, the reflected light beam emanating from the reflection area could be guided from said reflection area to an optical device or an object to be examined, in order to scan this. The optical device or the object to be examined can thus be passed by light beams that form a large overall scan angle. An object to be examined can be examined by a type of wide-angle lens.

A detector or only one detector could be provided, which detects a light beam returning from the optical device or the object to be examined or detects a signal of the returning light beam. The apparatus can have a compact structure should only one detector be used. The returning light beam can be scattered and/or reflected off the object to be examined.

The light beam returning from an object to be examined could be guided to the reflection area, from there said light beam could be guided by the optical means and from there said light beam could be guided to the reflection area again, in order to be guided from the latter to the detector. This allows an object to be examined to be scanned with high luminous intensity.

A stationary light beam emanating from the light source and a light beam returning to the detector could be guided in parallel and/or collinear fashion. This makes the structure of the apparatus compact and avoids the use of deflection means. Furthermore, the outgoing light beam and the returning light beam can made to interfere relatively easily, in order to optionally also capture OCT images.

The optical means could comprise at least two deflection mirrors. Deflection mirrors allow light beams to be deflected around corners reliably and with as little loss of intensity as possible. The overall scan angle can be easily set by a suitable arrangement and spacing of the deflection mirrors relative to each other.

The optical means could comprise at least two lenses or four lenses. In each case two lenses can be arranged confocal to each other, thus allowing a confocal structure of the apparatus.

The optical means could comprise at least one curved mirror. Such a mirror can firstly deflect light and at the same time focus it on a focal point.

The scanning device could comprise an X-scanning device or be designed as such, with a Y-scanning device additionally being provided. This makes it possible to scan an object areally, i.e. laterally. A two-dimensional representation of the object being examined can be performed.

The Y-scanning device could be designed as a deflection mirror, by means of which the outgoing reflected light beam, following the reflection thereof off two preceding deflection mirrors, can be guided back to the reflection area again as an actively returned light beam, in order to be guided from there to an optical device or to an object to be examined, in order to scan this in the Y-direction. This construction allows the two scanning devices to be used simultaneously, and the object can be scanned simultaneously in the X- and Y-directions. In particular, X- and Y-scanners or scanning devices are imaged onto each other on account of this construction, resulting in an ideal scanning pupil.

Light incident on the Y-scanning device could be guided through a third lens before being incident on the Y-scanning device, and light emanating from the Y-scanning device could be guided through a fourth lens. This allows the third lens to be arranged confocally to a first lens and the fourth lens to be arranged confocally to a second lens.

Against this background, at least two lenses could be arranged confocally to each other and/or arranged confocally to each other with the interposition of a deflection mirror or of two deflection mirrors. By using deflection mirrors, the confocally arranged lenses can be arranged in a particularly compact manner relative to each other. The coinciding focal points of two lenses used can be located slightly next to or on a deflection mirror.

By means of the apparatus described here, it is possible to adjust a scan angle by way of 4F imaging of a scan area onto itself. A further scan axis, which can be realized by a Y-scanning device, can be integrated into the apparatus and can be imaged onto the X-scanning device. This results in an optimal scanning pupil of the entire scanning system without optical offset. A compact intermediate image can be used for position feedback. A main optics unit or an optical device can optionally be simplified thereby. In addition, the apparatus described here brings about an increase in the image quality by way of a higher luminous intensity.

What can be realized by the apparatus described here is that the four times the amount of light emerges from an examined eye, and so a double scan is possible. There is a better numerical aperture or better optical resolution. Lower lens reflections occur on account of a larger possible beam diameter. The apparatus described here requires only one detector. It is not necessary to stitch multiple frames. No overlap of individual images is required. A shorter integration time at the detector can be realized with the same resolution. Multiple optical components can be introduced into a beam path. Scan angle enlargement is achieved by multiple scans.

In the drawing:

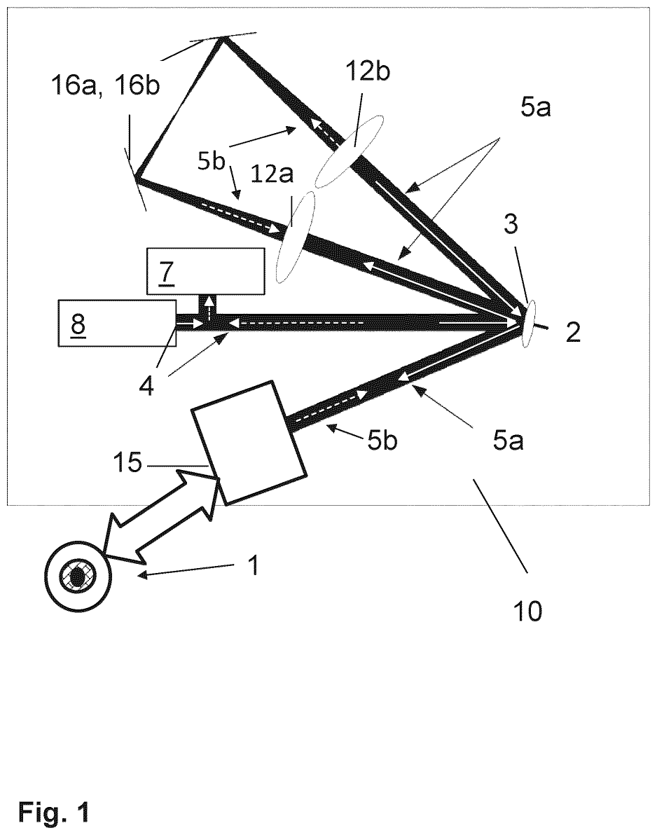

FIG. 1 shows a schematic illustration of the functional structure of an apparatus with a static or non-deflected scanning device,

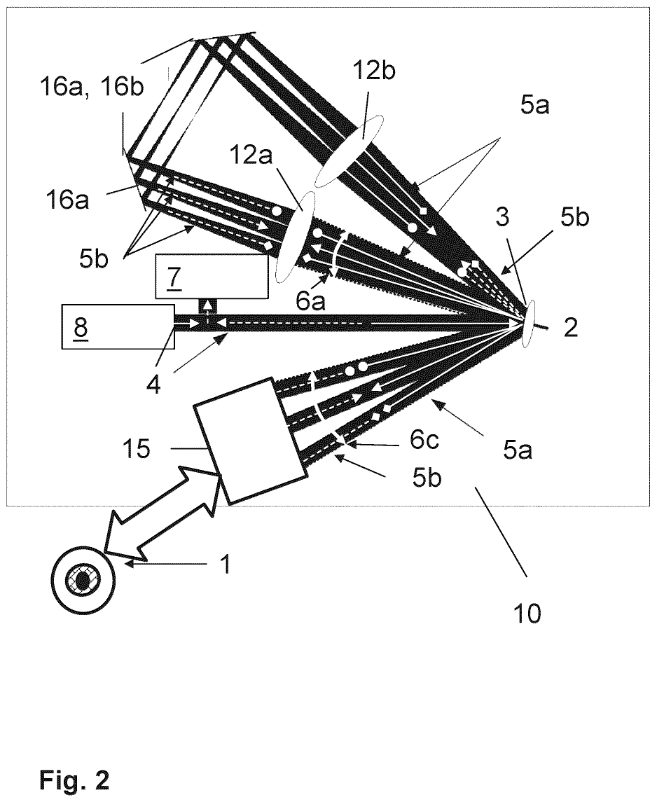

FIG. 2 shows the functional structure according to FIG. 1, wherein a scanner deflection is depicted schematically in order to represent the overall scan angle,

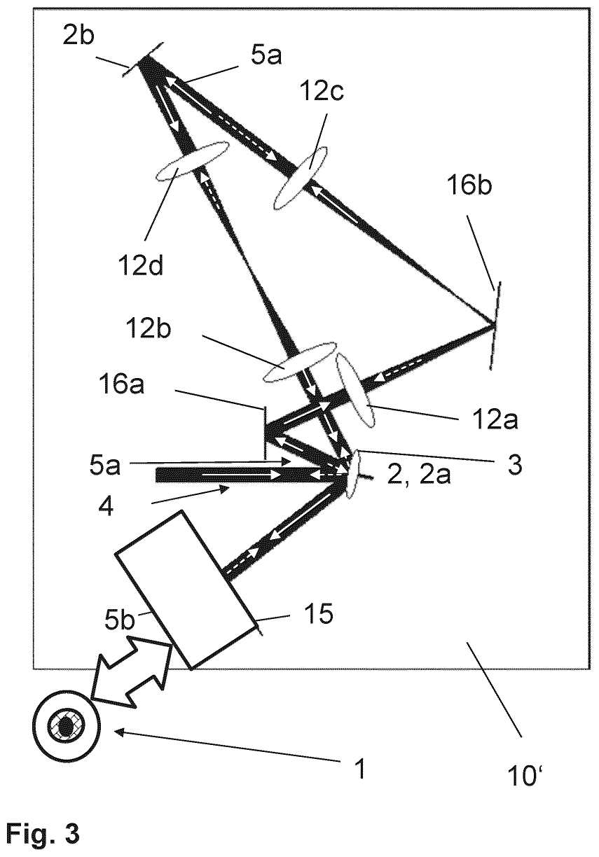

FIG. 3 shows a functional structure of a further apparatus, in which an additional Y-scan axis is realized, with a static scanning means, and

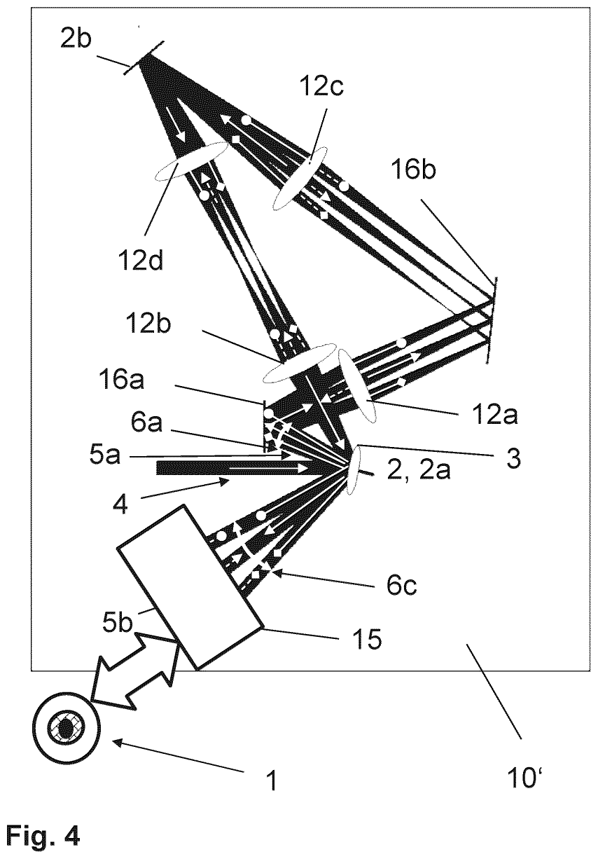

FIG. 4 shows the functional structure according to FIG. 3 with the additional Y-scan axis, wherein a scanner deflection is depicted schematically in order to represent the overall scan angle.

FIGS. 1 and 2 schematically show an apparatus 10 for examining an object or for scanning an eye 1, comprising a scanning device 2 having a reflection area 3, onto which at least one light beam 4 emitted by a light source 8 can be steered, with a reflected light beam 5a emanating from the reflection area 3 due to the incident light beam 4 being pivotable over a first angular range 6a by means of the scanning device 2 when the reflection area 3 is deflected.

In this respect, the incident light beam 4 is reflected and pivoted over the first angular range 6a as outgoing reflected light beam 5a. This is depicted in FIG. 2 by means of a schematically illustrated scanner deflection. The outgoing reflected light beam 5a is represented by a solid white line. In this respect, the outgoing reflected light beam 5a is a singly scanned light beam.

Optical means are provided, by means of which the outgoing reflected light beam 5a is pivotable over a second angular range 6c, which is larger than the first angular range 6a, after passing through said means. Specifically, the second angular range 6c is twice as large as the first angular range 6a and represents the overall scan angle of the apparatus. This is also depicted in FIG. 2.

The light beam 5a pivoted over the second angular range 6c is a twice-scanned light beam insofar as it is initially reflected off the reflection area 3 and guided to the optical means, then actively returned to the same location in the reflection area 3 and then pivoted from there to an optical device 15, in particular a main optics device, or to an object to be examined by way of the second angular range 6c, specifically the overall scan angle.

In this respect, FIG. 2 shows in concrete terms how the first angular range 6a can be converted into the second angular range 6c when the reflection area 3 of the scanning device 2 is deflected. Light beams 5a that are incident on and leave the reflection area 3 at different angles are illustrated schematically with different ends, the ends comprising a rhombus, a circle and an arrowhead.

Each reflected light beam 5a emanating from the reflection area 3 in the direction of the optical means can be guided from the optical means back to the reflection area 3 as an actively returned light beam 5a, and from there said reflected light beam can be pivoted over the second angular range 6c. This is illustrated by the fact that each actively returned light beam 5a is also represented by a solid white line.

In this respect, the outgoing reflected light beam and actively returned light beam 5a is first a singly scanned light beam and is then converted into a twice scanned light beam 5a when it is pivoted over the second angular range 6c.

A light beam 4 that is incident on the reflection area 3 and can also be referred to as a stationary light beam can be guided to the reflection area 3 at a different angle than an actively returned light beam 5a. The reflected light beam 5a emanating from the reflection area 3 is guided in a loop, as it were, and leaves the reflection area 3 first, in order to be incident thereon again after passing through the optical means and be guided from there to an optical device 15 or to an object to be examined.

After passing through the optical means, the reflected light beam 5a emanating from the reflection area 3 is guided from the reflection area 3 to an optical device 15 or an object to be examined, in order to scan this.

Specifically, only one detector 7 is provided, which detects a light beam 5b returning from the optical device 15 or the object to be examined or detects a signal of the returning light beam 5b. The returning beam 5b is a light beam backscattered or reflected off the object to be examined.

Light beams returning from the optical device 15 or the object to be examined are represented by dashed white lines.

Returning light beams 5b that are incident on the reflection area 3 and leave the latter again at different angles are illustrated schematically with different ends, the ends comprising a rhombus, a circle and an arrowhead.

Each returning light beam 5b can be guided to the reflection area 3, from there said light beam can be guided by the optical means and from there said light beam can be guided to the reflection area 3 again, in order to be guided from the latter to the detector 7.

Thus, a light beam 4 emanating from the light source 8 can be made to interfere with a returning light beam 5b, especially when a reference arm is present, in order to examine structures. Light beams 4 emanating from the light source 8 and light beams 5b actively returned to the detector 7 can be guided in parallel and/or collinear fashion.

FIGS. 1 and 2 show that the optical means comprise at least two deflection mirrors 16a, 16b. A reflected light beam 5a emanating from the reflection area 3 to the optical means is deflected by the deflection mirrors 16a, 16b around two angles or corners and guided to the reflection area 3 again as an actively returned light beam 5a.

The optical means further comprise two lenses 12a, 12b, which are arranged confocally in such a way that their focal points are located between the deflection mirrors 16a, 16b and on each other. As a result, the apparatus exhibits a confocal structure. Specifically, a first lens 12a and a second lens 12b are provided, specifically only two lenses.

FIGS. 3 and 4 show a further apparatus 10′ for examining an object or for scanning an eye 1, comprising a scanning device 2 with an X-scanning device 2a having a reflection area 3, onto which at least one light beam 4 emitted by a light source 8 (not shown here) can be steered, with a reflected light beam 5a emanating from the reflection area 3 due to the incident light beam 4 being pivotable over a first angular range 6a by means of the scanning device 2, 2a.

Analogously to FIG. 2, an outgoing reflected light beam 5a is shown using a solid white line.

Optical means are provided, by means of which the outgoing reflected light beam 5a is pivotable over a second angular range 6c, which is larger than the first angular range 6a, after passing through said means.

Specifically, the second angular range 6c is twice as large as the first angular range 6a and represents the overall scan angle of the apparatus.

The reflected light beam 5a emanating from the reflection area 3 in the direction of the optical means can be guided as an actively returned light beam 5a from the optical means back to the reflection area 3, and from there said reflected light beam can be pivoted over the second angular range 6c. This is illustrated by the fact that the actively returned light beam 5a is also represented by a solid white line.

A light beam 4 that is incident on the reflection area 3 can be guided to the reflection area 3 at a different angle than an actively returned light beam 5a. The light beam 5a is guided in a loop, as it were, and leaves the reflection area 3 first, in order to be incident thereon again after passing through the optical means and be guided from there to an optical device 15 or to an object to be examined.

After passing through the optical means, the reflected light beam 5a emanating from the reflection area 3 is guided as an actively returned light beam 5a from the reflection area 3 to an optical device 15 or an object to be examined, in order to scan this.

Specifically, only one detector 7 (not depicted here) is provided, which detects a light beam 5b returning from the optical device 15 or the object to be examined or detects a signal of the returning light beam 5b.

Light beams 5b returning from the optical device 15 or the object to be examined are represented by dashed white lines. Each returning light beam 5b can be guided to the reflection area 3, from there said light beam can be guided by the optical means and from there said light beam can be guided to the reflection area 3 again, in order to be guided from the latter to the detector 7.

Thus, a light beam 4 that emanates from the light source 8 and may also be referred to as a stationary light beam can in particular be made to interfere with a returning light beam 5b, in order to examine structures. A light beam 4 emanating from the light source 8 and a light beam 5b actively returned to the detector 7 can be guided in parallel and/or collinear fashion.

FIGS. 3 and 4 show that the optical means comprise two deflection mirrors 16a, 16b, which deflect the outgoing reflected light beam 5a and the returning light beam 5b around two angles or corners in each case.

The reflected light beam 5a emanating from the reflection area 3 to the optical means is deflected by a first deflection mirror 16a through a first lens 12a to a second deflection mirror 16b. From the second deflection mirror 16b, the outgoing reflected light beam 5a is guided through a further, third lens 12c.

The first lens 12a and the further, third lens 12c are arranged confocally in such a way that their focal points are located on top of each other slightly next to or on the second deflection mirror 16b.

From the further, third lens 12c, the outgoing reflected light beam 5a is steered to a Y-scanning device 2b, which is assigned to the scanning device 2 and/or electronically connected to the latter for simultaneous control. The scanning device 2 therefore comprises an X-scanning device 2a and additionally the Y-scanning device 2b. The X-scanning device 2a operates faster than the Y-scanning device 2b.

The Y-scanning device 2b is designed as a deflection mirror, by means of which the outgoing reflected light beam 5a, following the reflection off the two preceding deflection mirrors 16a, 16b, can be guided back to the reflection area 3, in order to be guided from there to the optical device 15 or to the object to be examined, in order to scan this in the Y-direction.

Both the outgoing reflected light beam 5a and the returning light beam 5b are therefore each deflected around three angles or corners.

From the Y-scanning device 2b, the outgoing reflected light beam 5a is led back to the reflection area 3 again as an actively returned light beam 5a after it has passed first through a fourth lens 12d and subsequently through a second lens 12b.

The second lens 12b and the fourth lens 12d are arranged confocally in such a way that their focal points are located on one another between said lenses. The actively returned light beam 5a is then guided from the X-scanning device 2a to the optical device 15 or the object to be examined.

In this respect, FIG. 4 shows, in a manner analogous to FIG. 2, the scanner deflection of the scanning device 2.

In FIGS. 3 and 4, the basic structure of the device according to FIGS. 1 and 2 is augmented by an additional scan mirror, specifically the Y-scanning device 2b. The Y-scanning device 2b is imaged on the X-scanning device 2a by way of 4F imaging such that an optimal scan pupil without optical offset arises. In addition, a complete, compact X-Y-intermediate image is available, which can be used for position feedback.

FIG. 2 and FIG. 4 each show the functional structure of the apparatuses on the basis of schematically illustrated scanner deflections.

Imaging need not necessarily be brought about by way of optical lenses as shown here. Alternatively, other optical components could be used, e.g. curved mirrors.

The principle of the apparatuses described here is as follows:

A light beam 4 is incident on a scanning device 2 and is variably deflected or scanned thereby. A 4F imaging setup comprising two lenses 12a, 12b and two deflection mirrors 16a, 16b is used to image the scan area or the impinged reflection area of the scanning device 2 onto itself.

On account of the deflection mirrors 16a, 16b, the returning, actively returned light beam 5a is incident on the scanning device 2 at a different angle than the outgoing reflected light beam 5a. Thus, the light beam 5a passes twice over the same scan area, whereby the overall scan angle 6c is doubled.

The ability to increase the overall scan angle 6c while maintaining the same light beam diameter in this manner offers various advantages. Thus, the gain, specifically the product of angle times area, can be used to realize faster systems with higher scan rates or a higher light output.

This is illustrated by the following further examples, which are intended to explain the effect of the invention described here on existing products:

### Example 1: Enhanced Luminous Intensity/Image Quality

CRS 4 kHz, 10 mm scan area, +/−10 degrees optical scan angle
60°, 4 kHz scan system

| | Conventional | Two-fold |
|---|---|---|
| | scanner | scanner |
| Frequency X-scanner [kHz] | 4 | 4 |
| Scan area diameter [mm] | 9 | 9 |
| Optical overall scan angle [deg.] | 20 | 40 |
| Target angle at the objective [deg.] | 60 | 60 |
| Optical scaling | 3 | 1.5 |
| Pupil on the eye diameter | 3 | 6 |

### Example 2: Increased Scan Speed

30°, 12 kHz scan system

| | Conventional | Two-fold |
|---|---|---|
| | scanner | scanner |
| Frequency X-scanner [kHz] | 8 | 12 |
| Scan area diameter [mm] | 5 | 5 |
| Optical overall scan angle [deg.] | 20 | 20 (2 × 10°) |
| Target angle at the objective [deg.] | 30 | 30 |
| Optical scaling | 1.5 | 1.5 |
| Pupil on the eye diameter | 3.33 | 3.33 |

The technology described herein can be used in a wide-angle system. The high luminous intensity obtained thereby results in a better image quality. Alternatively, an increased scan speed can also be realized. The aforementioned technical features are important prerequisites for the operability of an apparatus.

### LIST OF REFERENCE SIGNS

1 Eye or object to be examined
2 Scanning device
2a X-scanning device of 2
2b Y-scanning device of 2
3 Reflection area of 2, 2a
4 Light beam from 8 incident on 3
5a Light beam emanating from 3 and actively returned to 3
5a Light beam from 3 pivoted over 6c
5b Light beam returning from 1
6a First angular range of 5a
6c Second angular range of 5a
7 Detector
8 Light source
10, 10′ Apparatus
12a-12d First to fourth lens
15 Optical device or main optics unit
16a First deflection mirror
16b Second deflection mirror

1. An apparatus (10, 10′) for examining an object or for scanning an eye (1), comprising a scanning device (2, 2a) having a reflection area (3), onto which at least one light beam (4) emitted by a light source (8) can be steered, with a reflected light beam (5a) emanating from the reflection area (3) due to the incident light beam (4) being pivotable over a first angular range (6a) by means of the scanning device (2),
characterized in that optical means are provided, by means of which the outgoing reflected light beam (5a) is pivotable over a second angular range (6c), which is larger than the first angular range (6a), after passing through said means.

2. The apparatus as claimed in claim 1, characterized in that the second angular range (6c) is twice as large as the first angular range (6a).

3. The apparatus as claimed in claim 1 or 2, characterized in that the reflected light beam (5a) emanating from the reflection area (3) can be guided from the optical means back to the reflection area (3) and can be pivoted from there over the second angular range (6c).

4. The apparatus as claimed in claim 3, characterized in that the incident light beam (4) can be guided to the reflection area (3) at a different angle than the actively returned light beam (5a).

5. The apparatus as claimed in any of claims 1 to 4, characterized in that after passing through the optical means, the outgoing reflected light beam (5a) can be guided from the reflection area (3) to an optical device (15) or to an object to be examined, in order to scan this.

6. The apparatus as claimed in claim 5, characterized in that a detector (7) or only one detector (7) is provided, which detects a light beam (5b) returning from the optical device (15) or the object to be examined or detects a signal of the returning light beam (5b).

7. The apparatus as claimed in claim 6, characterized in that the returning light beam (5b) can be guided to the reflection area (3), from there said light beam can be guided by the optical means and from there said light beam can be guided to the reflection area (3) again, in order to be guided from the latter to the detector (7).

8. The apparatus as claimed in claim 7, characterized in that the outgoing light beam (4) and the light beam (5b) returning to the detector (7) can be guided in parallel and/or collinear fashion.

9. The apparatus as claimed in any of the preceding claims, characterized in that the optical means comprise at least two deflection mirrors (16a, 16b).

10. The apparatus as claimed in any of the preceding claims, characterized in that the optical means comprise at least two lenses (12a, 12b) or four lenses (12a, 12b, 12c, 12d).

11. The apparatus as claimed in any of the preceding claims, characterized in that the optical means comprise at least one curved mirror.

12. The apparatus (10′) as claimed in any of the preceding claims, characterized in that the scanning device (2) comprises an X-scanning device (2a) or is designed as such, with a Y-scanning device (2b) additionally being provided.

13. The apparatus as claimed in claim 12, characterized in that the Y-scanning device (2b) is designed as a deflection mirror, by means of which the outgoing reflected light beam (5a), following the reflection off two preceding deflection mirrors (16a, 16b), can be guided back to the reflection area (3), in order to be guided from there to an optical device (15) or to an object to be examined, in order to scan this in the Y-direction.

14. The apparatus as claimed in claim 12 or 13, characterized in that light incident on the Y-scanning device (2b) can be guided through a lens (12c) or third lens (12c), and light emanating from the Y-scanning device (2b) can be guided through a further lens (12d) or fourth lens (12d).

15. The apparatus as claimed in any of the preceding claims, characterized in that at least two lenses (12a, 12b, 12c, 12d) are arranged confocally to each other and/or arranged confocally to each other with the interposition of a deflection mirror (16b) or of two deflection mirrors (16a, 16b).
